(19) United States
(12) Patent Application Publication
(10) Pub. No.: US 2024/0374058 A1
(43) Pub. Date: Nov. 14, 2024

(54) ANTI-SNORING PILLOW

(71) Applicant: Aiworks Global, Inc., McKinney, TX (US)

(72) Inventor: Daohu Han, Brea, CA (US)

(21) Appl. No.: 18/316,154

(22) Filed: May 11, 2023

(51) Int. Cl. A47G 9/10; A61F 5/56

(52) U.S. Cl. CPC A47G 9/1027; A61F 5/56

(57) ABSTRACT

Provided herein is a system for snoring reduction of a user, having an anti-snoring pillow, a mobile application, a processor, and a database server; wherein the system is configured to perform operations comprising detecting snoring, and intervening with a position of the user's head until the snoring is reduced; the anti-snoring pillow housing air bags, a head position sensor, and a microphone board for the detection of snoring, wherein the mobile application is accessible by the user on a mobile device; wherein the processor is configured to perform operations comprising detecting snoring, intervening with the user's head position by inflating or deflating one or more of the air bags, analyzing patterns from the user, comparing the patterns from the user before and after the intervention, and utilizing artificial intelligence learning to optimize the settings based on the analysis and comparison for achieving a desired pattern from the user.

### BACKGROUND OF INVENTION

### 1. Field of the Invention

The invention relates generally to improvement of sleep quality, and more particularly to smart pillow devices for reducing snoring.

### 2. Description of the Related Art

Generally, people who snore during a sleeping session may wish for various reasons to reduce or eliminate their snoring entirely. An anti-snoring pillow designed to automatically adjust a user's head position may help to alleviate a user's snoring problems, but many existing pillows may not be suitable for different kinds of users. For example, some pillows may not be capable of making the kind of head positioning adjustments that would benefit all users. As an example, different users will have different height requirements for their pillows, and some pillows may not provide the heights that different users need.

Furthermore, users will have various habits and use environments. Individuals with differing snoring patterns may require different or customized head intervention partners. It is therefore difficult to meet all the needs of different users using a pillow designed with a single fixed height. Additionally, some existing pillows may only be provided with a single control mode, which cannot meet the diverse needs of various users. Therefore, there is a need for a solution to these problems.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

Provided herein are anti-snoring pillows comprising an air pump box, having a variety of noise reduction structures to effectively suppress noise, and create a comfortable sleeping environment. In some embodiments, the air pump box comprises a sponge tier, a metallic conduit tier, an intensification sponge tier, a plastic tier, a sound silencing box, and a shell. In some embodiments, two sound silencing boxes A and B are provided.

Provided herein are methods for detecting and reducing snoring of a user via a system, the system comprising an anti-snoring pillow, a mobile application for use with the anti-snoring pillow, a processor, and a database server, wherein the anti-snoring pillow, the mobile application, and the database server are in communication with each other by WiFi; wherein the mobile application and the anti-snoring pillow can connect to each other by Bluetooth; wherein the system is configured to perform operations comprising detecting snoring, massaging a head of the user, and intervening with a position of the user's head until the snoring is reduced; the anti-snoring pillow comprising: a pillow component having an interior for housing a plurality of air bags, a head position sensor, a microphone board for the detection of snoring, a control box having an air pump, and a printed circuit board assembly, and an outlet pipe for connecting the air pump to the plurality of air bags; wherein the mobile application is accessible by the user on a mobile device, and the mobile application comprises controls for adjusting settings of the operations performed by the system; wherein the processor is configured to perform operations comprising utilizing artificial intelligence learning to analyze patterns from the user and optimizing the settings based on the performed analysis; wherein the database server comprises cloud storage for data received from the mobile application and the anti-snoring pillow; the methods comprising: detecting a first occurrence of snoring from the user; intervening with the user's head position by inflating or deflating one or more of the air bags of the plurality of air bags; analyzing the patterns from the user, wherein the patterns comprise presence or absence of snoring, volume of snoring, and frequency of snoring; comparing the patterns from the user before and after the intervention; optimizing the settings based on the analyzing and the comparing steps for achieving a desired pattern from the user; and storing the data related to the analysis, comparison, and the optimization in the database server.

Provided herein are systems for snoring reduction of a user, comprising an anti-snoring pillow, a mobile application, a processor, and a database server; wherein the anti-snoring pillow, the mobile application, the processor, and the database server are in communication with each other by WiFi; wherein the mobile application and the anti-snoring pillow can connect to each other by Bluetooth; wherein the system is configured to perform operations comprising detecting snoring, massaging a head of the user, and intervening with a position of the user's head until the snoring is reduced; the anti-snoring pillow comprising: a pillow component having an interior for housing a plurality of air bags, a head position sensor, a microphone board for the detection of snoring, a control box having an air pump, and a printed circuit board assembly, and an outlet pipe for connecting the air pump to the plurality of air bags; wherein the mobile application is accessible by the user on a mobile device, and the mobile application comprises controls for adjusting settings of the snoring reduction, the settings sensitivity of the detection of snoring, intensity of the massage, and sensitivity of the intervention of the position of the user's head; wherein the processor is configured to perform operations comprising detecting snoring, intervening with the user's head position by inflating or deflating one or more of the air bags of the plurality of air bags, analyzing patterns from the user, comparing the patterns from the user before and after the intervention, and comprising utilizing artificial intelligence learning to optimize the settings based on the analysis and comparison for achieving a desired pattern from the user; and wherein the database server comprises cloud storage for data received from the mobile application and the anti-snoring pillow.

Provided herein is a mobile application for snoring reduction of a user, the mobile application being accessible through a mobile device and configured to communicate with an anti-snoring pillow, a processor, and a database server; wherein the anti-snoring pillow, the mobile application, and the database server are in communication with each other by WiFi; wherein the mobile application and the anti-snoring pillow can connect to each other by Bluetooth; wherein the mobile application is configured to send instructions to the anti-snoring pillow for performing operations comprising detecting snoring, massaging a head of the user, and intervening with a position of the user's head until the snoring is reduced; the anti-snoring pillow comprising: a pillow component having an interior for housing a plurality of air bags, a head position sensor, a microphone board for the detection of snoring, a control box having an air pump, and a printed circuit board assembly, and an outlet pipe for connecting the air pump to the plurality of air bags; the mobile application comprising controls for adjusting settings of the operations performed by the system; and wherein the processor is configured to perform operations comprising detecting snoring, intervening with the user's head position by inflating or deflating one or more of the air bags of the plurality of air bags, analyzing patterns from the user, comparing the patterns from the user before and after the intervention, and comprising utilizing artificial intelligence learning to optimize the settings based on the analysis and comparison for achieving a desired pattern from the user.

In some embodiments, the anti-snoring pillows provided herein may comprise adjustability, such that the pillow can be adjusted to various heights suitable for a variety of users. In some embodiments, the pillows provided herein comprise three levels of height adjustment.

In some embodiments, the anti-snoring pillows provided herein may comprise multiple control modes. For example, the pillow may be controlled by a mobile app, and by a button on the pillow, such that users of all technological comfort levels may user the pillow's functions. In some embodiments, the pillow can be controlled remotely or operated offline. In some embodiments, data collected by the anti-snoring pillow may be transmitted to the cloud through WiFi. In some embodiments, artificial intelligence (AI) learning is utilized for improving the snoring detection system of the pillows disclosed herein. In such embodiments, the detection system may be improved through AI learning to distinguish between snoring and other noise, such as environmental background noise, and to distinguish between snoring from the person using the pillow or from another nearby person.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1A depicts an exploded view of an exemplary anti-snoring pillow and various interior components of the anti-snoring pillow, according to an aspect.

FIG. 1B is a list of the components shown in FIG. 1A, and exemplary quantities of each listed component, according to an aspect.

FIGS. 1C-1D depict two side perspective views of an anti-snoring pillow comprising a pillow component 120, and a control box 121, according to an aspect.

FIG. 2 depicts a top perspective cutaway view of the control box of an anti-snoring pillow, according to an aspect.

FIG. 3 depicts a side view of anti-snoring pillow showing the adjustable height of the pillow, according to an aspect.

FIG. 4A depicts a top view of the control box 121 of an anti-snoring pillow, according to an aspect.

FIGS. 4B-4C depict two examples of user interfaces that may be accessed by a user through a mobile application provided on an electronic device 130, according to an aspect.

FIG. 4D illustrates a diagrammatic view of a computer system for analyzing data received from a pillow component 120 by a control box 121, and storage of the data in a cloud server 122, according to an aspect.

FIG. 5 is a flow chart depicting an example of how a mobile application for an anti-snoring pillow can be connected to a network and used for performing tasks, according to an aspect.

FIG. 6 depicts an exploded side view of the components of a pillow component of an anti-snoring pillow device, according to an aspect.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

It should be understood that, for clarity of the drawings and of the specification, some or all details about some structural components or steps that are known in the art are not shown or described if they are not necessary for the invention to be understood by one of ordinary skills in the art.

As used herein and throughout this disclosure, the term “mobile device” refers to any electronic device capable of communicating across a mobile network. A mobile device may have a processor, a memory, a transceiver, an input, and an output. Examples of such devices include cellular telephones, personal digital assistants (PDAs), portable computers, etc. The memory stores applications, software, or logic. Examples of processors are computer processors (processing units), microprocessors, digital signal processors, controllers and microcontrollers, etc. Examples of device memories that may comprise logic include RAM (random access memory), flash memories, ROMS (read-only memories), EPROMS (erasable programmable read-only memories), and EEPROMS (electrically erasable programmable read-only memories). A transceiver includes but is not limited to cellular, GPRS, Bluetooth, and Wi-Fi transceivers.

“Logic” as used herein and throughout this disclosure, refers to any information having the form of instruction signals and/or data that may be applied to direct the operation of a processor. Logic may be formed from signals stored in a device memory. Software is one example of such logic. Logic may also be comprised by digital and/or analog hardware circuits, for example, hardware circuits comprising logical AND, OR, XOR, NAND, NOR, and other logical operations. Logic may be formed from combinations of software and hardware. On a network, logic may be programmed on a server, or a complex of servers. A particular logic unit is not limited to a single logical location on the network.

Mobile devices communicate with each other and with other elements via a network, for instance, a cellular network. A “network” can include broadband wide-area networks, local-area networks, and personal area networks. Communication across a network can be packet-based or use radio and frequency/amplitude modulations using appropriate analog-digital-analog converters and other elements. Examples of radio networks include GSM, CDMA, Wi-Fi and BLUETOOTH® networks, with communication being enabled by transceivers. A network typically includes a plurality of elements such as servers that host logic for performing tasks on the network. Servers may be placed at several logical points on the network. Servers may further be in communication with databases and can enable communication devices to access the contents of a database. For instance, an authentication server hosts or is in communication with a database having authentication information for users of a mobile network. A “user account” may include several attributes for a particular user, including a unique identifier of the mobile device(s) owned by the user, relationships with other users, call data records, bank account information, etc. A billing server may host a user account for the user to which value is added or removed based on the user's usage of services. One of these services includes mobile payment. In exemplary mobile payment systems, a user account hosted at a billing server is debited or credited based upon transactions performed by a user using their mobile device as a payment method.

For the following description, it can be assumed that most correspondingly labeled elements across the figures (e.g., 105 and 205, etc.) possess the same characteristics and are subject to the same structure and function. If there is a difference between correspondingly labeled elements that is not pointed out, and this difference results in a non-corresponding structure or function of an element for a particular embodiment, example or aspect, then the conflicting description given for that particular embodiment, example or aspect shall govern.

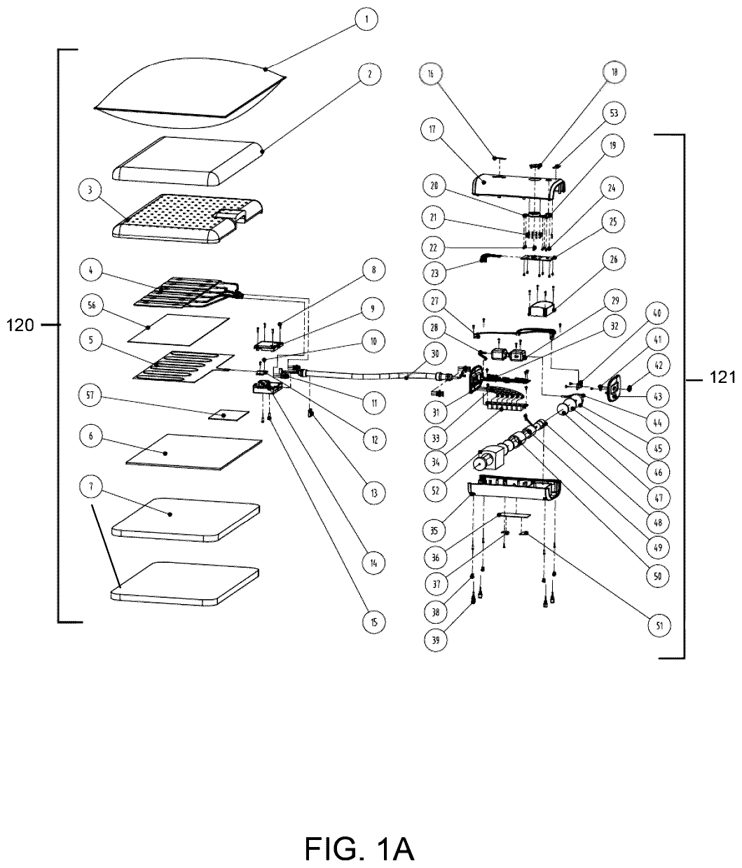

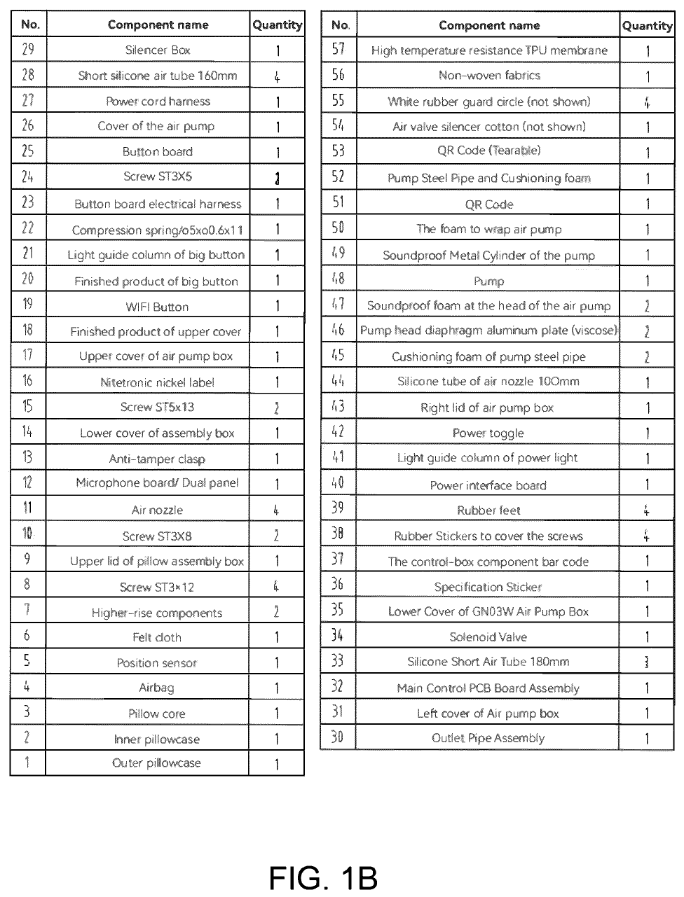

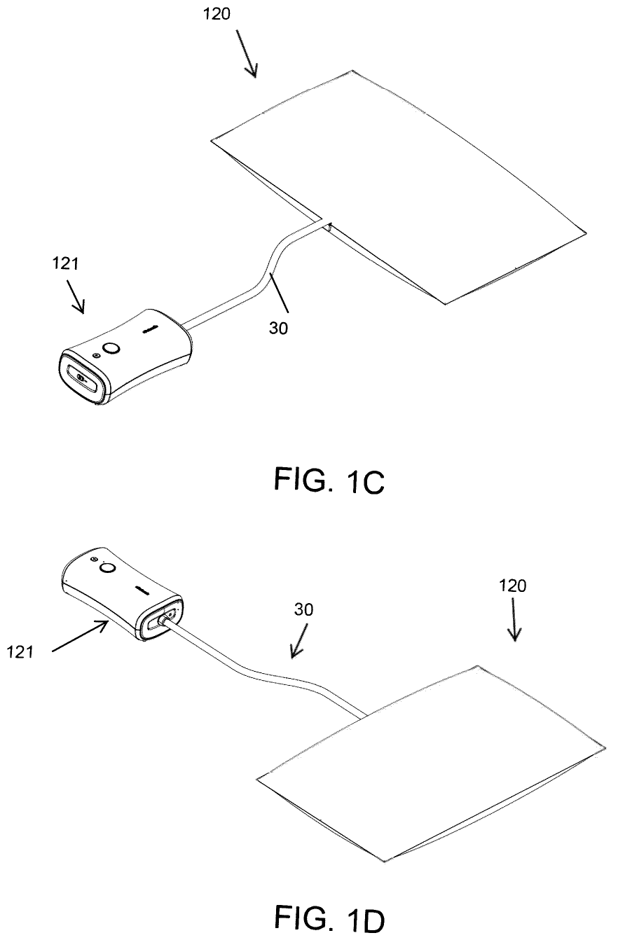

FIG. 1A depicts an exploded view of an exemplary anti-snoring pillow and various interior components of the anti-snoring pillow, according to an aspect. In some embodiments, the anti-snoring pillows provided herein comprise a pillow component 120, and a control box 121. Generally, the pillow component 120 comprises a pillow and air bags for adjusting the position of a user's head, and the control box 121 comprises elements for analyzing data relating to the user collected by the pillow component 120, for controlling the pillow component 120, and for commuting with other electronic devices.

FIG. 1B is a list of the components shown in FIG. 1A, and exemplary quantities of each listed component, according to an aspect. In some embodiments, the anti-snoring pillow comprises at least the following components: an outer pillowcase (also referred to herein as a jacket) 1, an inner pillowcase 2, a pillow core 3, a plurality of air bags 4, a head position sensor 5, a felt cloth 6, higher-rise components (also referred to herein as height adjusters) 7, an upper lid 9 of a pillow assembly box, screws 8 for the upper lid 9, a dual panel microphone board 12, screws 10 for the microphone board 12, an air nozzle 11, an anti-tamper clasp 13, a lower cover 14 of the pillow assembly box, screws 15 for the lower cover 14, a nickel label 16, an upper cover 17 of an air pump box, a finished product 18 of the upper cover 17, a WiFi control button 19, a finished product 20 of the control button 19, a light guide column 21, a compression spring 22, a button board 25, a button board electrical harness 23, screws 24 for the button board 25, an air pump cover 26, a power cord harness 27, a short silicone air tube 28 for a silencer box, a silencer box 29, an outlet pipe assembly (also referred to herein as a cord) 30, a left cover 31 of the air pump box, a main control 32 for a printed circuit board (PCB) board assembly, a silicone short air tube 33 for the air pump box, a solenoid valve 34, a lower cover 35 for the air pump box, a specification sticker 36, a control box component bar code 37, rubber stickers 38 for covering screws, rubber feet 39, a power interface board 40, a light guide column of power lights 41, a power toggle 42, a right lid of the air pump box 43, a silicone tube 44 for the air nozzle, cushioning foam 45 for a pump steel pipe, a pump head diaphragm aluminum plate 46, soundproof foam 47 for the head of the air pump, air pump 48, a soundproof metal cylinder 49 of the pump, foam 50 for wrapping the air pump, a QR code 51, a pump steel pipe and cushioning foam 52, a second QR code 52 (which may be removable or tearable), an air valve silencer cotton (not shown), white rubber guard circles (not shown), non-woven fabric 56, and a high temperature resistant thermoplastic polyurethan (TPU) membrane 57.

FIGS. 1C-1D depict two side perspective views of an anti-snoring pillow comprising a pillow component 120, and a control box 121, according to an aspect. In some embodiments, the pillow component 120 and the control box 121 are connected by a cord 30.

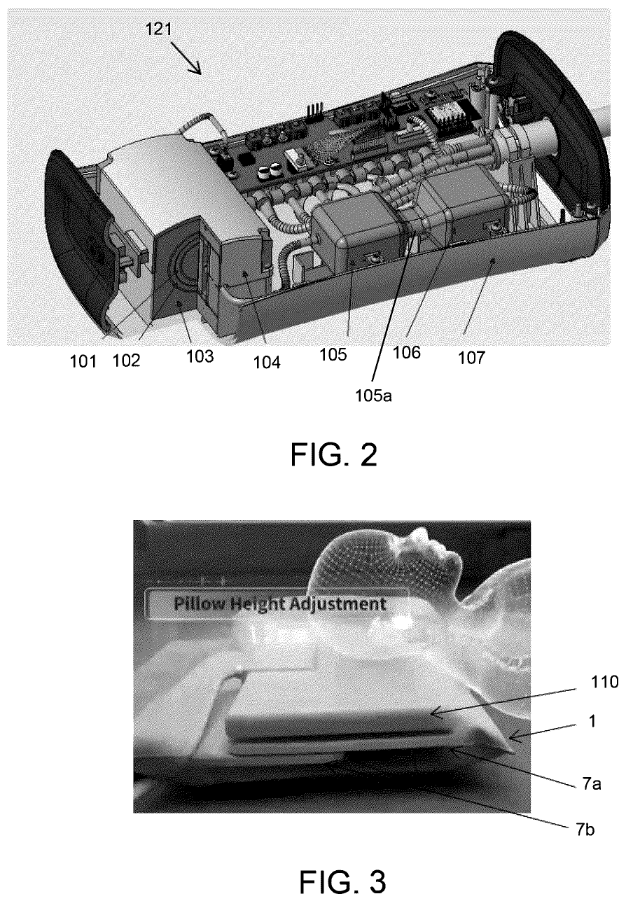

FIG. 2 depicts a top perspective cutaway view of the control box 121 of an anti-snoring pillow, according to an aspect. The control box may comprise the air pump system and electronic control system of the anti-snoring pillow. In some embodiments, the air pump box comprises a plurality of tiers or layers, wherein the terms “tier” and “layer” each refer to the various materials used for encasing the air pump. In some embodiments, the air pump box comprises a sponge tier 101, a metallic conduit tier 102, a thickened sponge tier 103, a plastic tier 104, a first sound silencing box 105 (also referred to herein as “sound silencing box A”), a second sound silencing box 106 (also referred to herein as “sound silencing box B”), and a shell 107. The air pump box may employ such various structures for noise reduction in order to effectively suppress noise, and create a comfortable sleeping environment.

In any of the anti-snoring pillows disclosed herein, the air pump itself may create some noise and vibration during use due to the sound of air flow and vibrations caused by the air pump. Therefore, the anti-snoring pillows disclosed herein employ a variety of measures on the air pump box for noise reduction. First, the air pump body may be coated with a sponge tier 101, wherein the sponge material can isolate some of the noise of the air pump. The sponge tier 101 may also act as a buffer between the air pump and the metallic conduit tier 102, which may be arranged exterior to the sponge layer 101. The metal conduit tier 102 may comprise a metal tube having a mass and density suitable for effectively inhibiting the majority of the vibrations from the air pump, and therefore can isolate most of the noise of the air pump. The metal conduit tier 102 may be coated with an intensification sponge tier 103, which can further isolate the noise of the air pump and also provide cushioning against the vibrations of the air pump. Exterior to the thickened sponge tier 103 may be a plastic tier 104, which is used to fix the thickened sponge tier 103 in place. A first sound silencing box 105 and a second sound silencing box 106 may be connected by a bridge 105a and allow air flow along their interiors, thus increasing the path of the air flow from the air pump. This may allow more time for the air to flow and move, and slow down within the silencing boxes, thereby reducing the noise of the air flow. Finally, the shell 107 provides an outer cover of the entire air pump box and provides a final barrier for dampening noise and vibrations.

FIG. 3 depicts a side view of anti-snoring pillow showing the adjustable height of the pillow, according to an aspect. In some embodiments, the anti-snoring pillows disclosed herein comprise a pillow core assembly 110, which contains the electronic components of the anti-snoring pillow, an outer pillowcase or pillow jacket 2, a first height adjuster 7a, and a second height adjuster 7b. In such embodiments, the anti-snoring pillow allows a user to adjust the pillow to their preferred height and has three heights to choose from. Using the pillow without either the first height adjuster 7a or the second height adjuster 7b allows the user to use the pillow at its lowest height. Inserting the first height adjuster 7a and the second height adjuster 7b allows the user to use the pillow at its highest height. Inserting only the first height adjuster 7a allows the user to use the pillow at a height between the lowest and the highest heights. Each of the height adjusters 7a and 7b can be inserted into the interior of the pillow jacket 1.

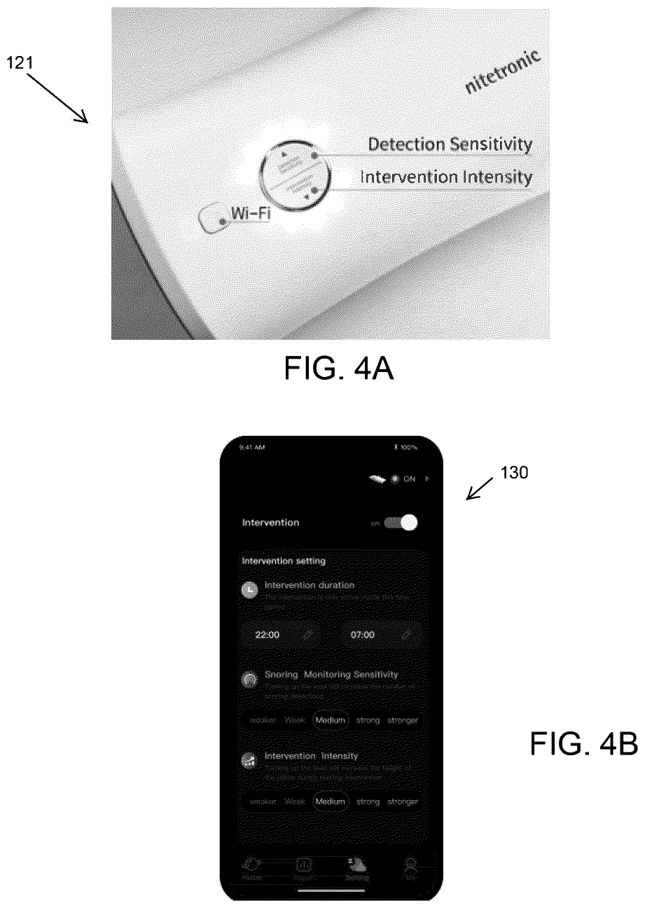

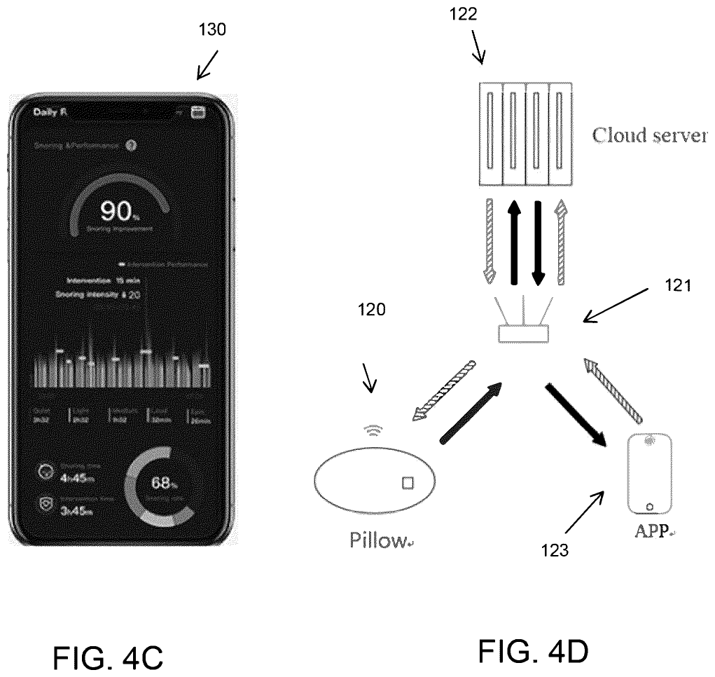

FIG. 4A depicts a top view of the control box 121 of an anti-snoring pillow, according to an aspect. The control box 121 may include buttons 122 for controlling the anti-snoring pillows disclosed herein. In some embodiments, the control box 121 includes buttons for on/off functions, for connecting to WiFi, for controlling the sensitivity of snoring detection, and for controlling the intensity of intervention or changing of the user's head position when snoring is detected.

FIGS. 4B-4C depict two examples of user interfaces that may be accessed by a user through a mobile application provided on an electronic device 130, according to an aspect. In some embodiments, the electronic device is a smart phone or any other suitable mobile device. A mobile application for controlling the anti-snoring pillows disclosed herein may be accessed by a user to adjust various controls. For example, as discussed when referring to the control box depicted in FIG. 4A, the mobile application can be used for on/off functions, for connecting the pillow to WiFi, for controlling the sensitivity of snoring detection, and for controlling the intensity of intervention when snoring is detected. It should be understood that the anti-snoring pillows disclosed herein may be provided with controls available on both the control box and a mobile application, such that users may use whichever controls are most comfortable or convenient for them.

FIG. 4D illustrates a diagrammatic view of a computer system for analyzing data received from a pillow component 120 by a control box 121, and storage of the data in a cloud server 122, according to an aspect. In some embodiments, the pillow 120 may receive data from a user during use, and communicate the data to the control box 121. A mobile application 123 may be used to communicate across a network such as a WiFi network and send controls to the control box 121. The data received and analyzed by the control box 121 can be stored in a cloud server 122, and data stored in the cloud server 122 can also be downloaded or received by the control box 121.

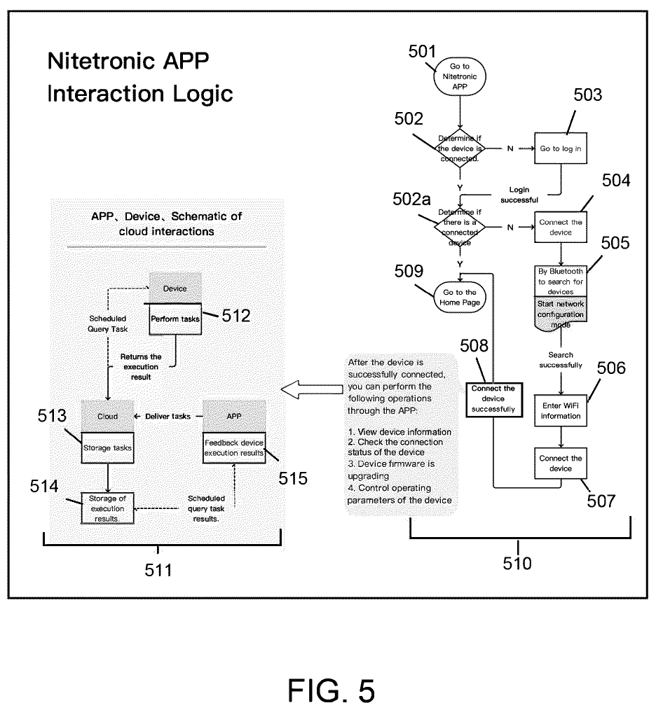

FIG. 5 is a flow chart depicting an example of how a mobile application for an anti-snoring pillow can be connected to a network and used for performing tasks, according to an aspect. Section 510 of the flow chart shows steps for a user connecting an anti-snoring pillow device to a WiFi connection via a mobile application. Section 511 of the flow chart shows steps for the mobile application, anti-snoring pillow, and cloud storage, for a variety of interactions. In some embodiments, a mobile application is accessed by a user (step 501) and the system may first detect whether the anti-snoring pillow device is connected (step 502). If no, the user may be prompted to log into a user account (step 503). After a user is successfully logged in, the application can determine if there is a connected device (step 502a). If yes, the user can be taken to a home page menu of the application (step 509). If no, the application will connect the anti-snoring pillow device (step 504). This step can be carried out by searching for nearby devices via Bluetooth (step 505) to start a network configuration mode. After successfully searching and locating a device, the user can be prompted to enter WiFi information (step 506), after which connection to the device is initiated (step 507). The device can then be connected successfully (step 508), and the user can be directed to a home page menu of the application (step 509).

After the above steps are carried out and the anti-snoring device is successfully connected to the mobile application via WiFi, a user may perform various tasks through the mobile application. These tasks include, but are not limited to, viewing device information, checking the connection status of the anti-snoring pillow device, upgrade or check upgrade status of the device firmware, and control the operating parameters of the device. For example, the app may deliver tasks to the cloud, and the anti-snoring pillow device may receive scheduled query tasks from the cloud. Scheduled tasks may, for example, comprise scheduling a period of time for detection and reduction of snoring. The pillow device can then perform tasks (step 512) and return the execution of these results to the cloud. The cloud can store tasks (step 513) and also store the execution results (step 514). The application may receive feedback from the anti-snoring pillow device (step 515) and send query tasks results to the cloud. As an example, sending query tasks may comprise the mobile application operating a function, and the command being transmitted to the anti-snoring pillow through the network by which the mobile application and anti-snoring pillow are connected. If the pillow receives the command and executes the command, the pillow will next inform the mobile application of the result through the network.

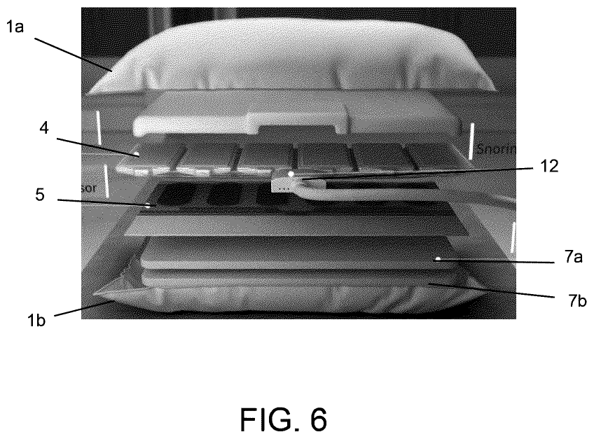

FIG. 6 depicts an exploded side view of the components of a pillow component of an anti-snoring pillow device, according to an aspect. It should be understood that the outer pillowcase 1 is shown in top and bottom halves 1a and 1b, respectively, for visual clarity; however, in some embodiments, the outer pillowcase 1 is provided as a single integrated unit with an opening such that the other elements of the pillow component can be placed inside. Shown in this view are air chambers (also referred to herein as air bags) 4, height adjusters 7a and 7b, a microphone board 12, which acts as a snoring recognition sensor, and a head position sensor 5. In some embodiments, the snoring sensor is a micro-electromechanical system (MEMS) sensor. In some embodiments, the head position sensor 5 is a capacitive head position sensor (CECS).

In some embodiments, the anti-snoring pillows disclosed herein provide a massage function which can effectively relieve neck fatigue. The air bags 4 may be provided in a plurality of air bags set in a row of six. In some embodiments, the anti-snoring pillows comprise one, two, three, four, five, six, seven, eight, nine, ten, or more air bags. In some embodiments, the air bags are in a horizontal row of six air bags. In some embodiments, a massage function is turned on automatically when the head position sensor detects that a user has put their head on the pillow. First, the pillow judges the head position of the user using the head position sensor 5, and then inflates the corresponding air bags with an air pump (as shown in FIG. 1A), according to a predetermined, specific massage logic. For example, when the head sensor detects that the head is in the middle position of the pillow, the system will start the air pump and the corresponding air valve, and push the third and fourth air bags alternatively according to a predetermined logic, to achieve massage. It should be understood that any suitable programs or systems for massage sequences of the air bags may be implemented. In some embodiments, new, updated, or improved massage functions or sequences may be downloaded to the mobile application for use with the anti-snoring pillow, and may be received by the pillow device through a WiFi connection.

In some embodiments, the anti-snoring pillows provided herein comprise dynamic adjustment capabilities such that the pillow can adjust a variety of settings as the device is in use. For example, the mobile application may be provided with an AI algorithm which allows for dynamic learning for each individual user. The algorithm can compare data from continued uses by a particular user. If it finds that the anti-snoring adjustments made by the pillow are not ideal or insufficient for reducing snoring of the user, it will automatically adjust parameters such as the anti-snoring adjustment intensity (i.e., movements made by the air bags due to level of inflation of the air bags). After adjusting the intensity, it will again compare the received data until finding the most suitable parameters for a particular user, so as to achieve an ideal anti-snoring effect.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims.

If present, use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the aspects, embodiments or examples shown should be considered as exemplars, rather than limitations on the apparatus or procedures disclosed or claimed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

If means-plus-function limitations are recited in the claims, the means are not intended to be limited to the means disclosed in this application for performing the recited function, but are intended to cover in scope any equivalent means, known now or later developed, for performing the recited function.

Claim limitations should be construed as means-plus-function limitations only if the claim recites the term “means” in association with a recited function.

If any presented, the claims directed to a method and/or process should not be limited to the performance of their steps in the order written, and one skilled in the art can readily appreciate that the sequences may be varied and still remain within the spirit and scope of the present invention.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples. Hence, the scope of the invention is defined by the accompanying claims and their equivalents. Further, each and every claim is incorporated as further disclosure into the specification.

What is claimed is:

1. A method for detecting and reducing snoring of a user via a system, the system comprising an anti-snoring pillow, a mobile application for use with the anti-snoring pillow, a processor, and a database server, wherein the anti-snoring pillow, the mobile application, and the database server are in communication with each other by WiFi;
wherein the mobile application and the anti-snoring pillow can connect to each other by Bluetooth;
wherein the system is configured to perform operations comprising detecting snoring, massaging a head of the user, and intervening with a position of the user's head until the snoring is reduced;
the anti-snoring pillow comprising:
a pillow component having an interior for housing a plurality of air bags, a head position sensor, a microphone board for the detection of snoring,
a control box having an air pump, and a printed circuit board assembly, and
an outlet pipe for connecting the air pump to the plurality of air bags;
wherein the mobile application is accessible by the user on a mobile device, and the mobile application comprises controls for adjusting settings of the operations performed by the system;
wherein the processor is configured to perform operations comprising utilizing artificial intelligence learning to analyze patterns from the user and optimizing the settings based on the performed analysis; wherein the database server comprises cloud storage for data received from the mobile application and the anti-snoring pillow; the method comprising:
detecting a first occurrence of snoring from the user;
intervening with the user's head position by inflating or deflating one or more of the air bags of the plurality of air bags;
analyzing the patterns from the user, wherein the patterns comprise presence or absence of snoring, volume of snoring, and frequency of snoring;
comparing the patterns from the user before and after the intervention;
optimizing the settings based on the analyzing and the comparing steps for achieving a desired pattern from the user; and
storing the data related to the analysis, comparison, and the optimization in the database server.

2. The method of claim 1, wherein the desired pattern is an absence of snoring.

3. The method of claim 1, wherein the artificial intelligence learning of the processor is used to compare data from continued uses of the system by the user of at least two sleeping sessions.

4. The method of claim 1, wherein the mobile application is configured to perform tasks comprising viewing information related to the anti-snoring pillow, upgrading firmware related to the anti-snoring pillow, and scheduling a period of time for detection and reduction of snoring.

5. The method of claim 1, wherein the settings adjustable via the mobile application comprises turning the anti-snoring pillow on or off, sensitivity of the detection of snoring, intensity of the massage, and sensitivity of the intervention of the position of the user's head.

6. The method of claim 1, wherein the control box comprises buttons for turning the anti-snoring pillow on or off, and adjusting the settings of the operations performed by the system.

7. A system for snoring reduction of a user, comprising an anti-snoring pillow, a mobile application, a processor, and a database server; wherein the anti-snoring pillow, the mobile application, the processor, and the database server are in communication with each other by WiFi; wherein the mobile application and the anti-snoring pillow can connect to each other by Bluetooth;
wherein the system is configured to perform operations comprising detecting snoring, massaging a head of the user, and intervening with a position of the user's head until the snoring is reduced;
the anti-snoring pillow comprising:
a pillow component having an interior for housing a plurality of air bags, a head position sensor, a microphone board for the detection of snoring,
a control box having an air pump, and a printed circuit board assembly, and
an outlet pipe for connecting the air pump to the plurality of air bags;
wherein the mobile application is accessible by the user on a mobile device, and the mobile application comprises controls for adjusting settings of the snoring reduction, the settings sensitivity of the detection of snoring, intensity of the massage, and sensitivity of the intervention of the position of the user's head; wherein the processor is configured to perform operations comprising detecting snoring, intervening with the user's head position by inflating or deflating one or more of the air bags of the plurality of air bags, analyzing patterns from the user, comparing the patterns from the user before and after the intervention, and comprising utilizing artificial intelligence learning to optimize the settings based on the analysis and comparison for achieving a desired pattern from the user; and
wherein the database server comprises cloud storage for data received from the mobile application and the anti-snoring pillow.

8. The system of claim 7, wherein the patterns from the user comprise presence or absence of snoring, volume of snoring, and frequency of snoring.

9. The system of claim 7, wherein the desired pattern is an absence of snoring.

10. The system of claim 7, wherein the artificial intelligence learning of the processor is used to compare data from continued uses of the system by the user of at least two sleeping sessions.

11. The system of claim 7, wherein the mobile application is configured to perform tasks comprising viewing information related to the anti-snoring pillow, upgrading firmware related to the anti-snoring pillow, and scheduling a period of time for detection and reduction of snoring.

12. The system of claim 7, wherein the settings adjustable via the mobile application comprises turning the anti-snoring pillow on or off, sensitivity of the detection of snoring, intensity of the massage, and sensitivity of the intervention of the position of the user's head.

13. The system of claim 7, wherein the control box comprises buttons for turning the anti-snoring pillow on or off, and adjusting settings of the snoring reduction, the settings sensitivity of the detection of snoring, intensity of the massage, and sensitivity of the intervention of the position of the user's head.

14. A mobile application for snoring reduction of a user, the mobile application being accessible through a mobile device and configured to communicate with an anti-snoring pillow, a processor, and a database server; wherein the anti-snoring pillow, the mobile application, and the database server are in communication with each other by WiFi;
wherein the mobile application and the anti-snoring pillow can connect to each other by Bluetooth;
wherein the mobile application is configured to send instructions to the anti-snoring pillow for performing operations comprising detecting snoring, massaging a head of the user, and intervening with a position of the user's head until the snoring is reduced;
the anti-snoring pillow comprising:
a pillow component having an interior for housing a plurality of air bags, a head position sensor, a microphone board for the detection of snoring,
a control box having an air pump, and a printed circuit board assembly, and
an outlet pipe for connecting the air pump to the plurality of air bags;
the mobile application comprising controls for adjusting settings of the operations performed by the system; and
wherein the processor is configured to perform operations comprising detecting snoring, intervening with the user's head position by inflating or deflating one or more of the air bags of the plurality of air bags, analyzing patterns from the user, comparing the patterns from the user before and after the intervention, and comprising utilizing artificial intelligence learning to optimize the settings based on the analysis and comparison for achieving a desired pattern from the user.

15. The mobile application of claim 14, wherein the database server comprises cloud storage for data received from the mobile application and the anti-snoring pillow.

16. The mobile application of claim 14, wherein the mobile application is configured to perform tasks comprising viewing information related to the anti-snoring pillow, upgrading firmware related to the anti-snoring pillow, and scheduling a period of time for detection and reduction of snoring.

17. The mobile application of claim 14, the settings adjustable via the mobile application comprises sensitivity of the detection of snoring, intensity of the massage, and sensitivity of the intervention of the position of the user's head.

18. The mobile application of claim 14, wherein the patterns from the user comprise presence or absence of snoring, volume of snoring, and frequency of snoring.

19. The mobile application of claim 14, wherein the desired pattern is an absence of snoring.

20. The mobile application of claim 14, wherein the artificial intelligence learning of the processor is used to compare data from continued uses of the system by the user of at least two sleeping sessions.
